(19) United States
(12) Patent Application Publication
(10) Pub. No.: US 2005/0186184 A1
(43) Pub. Date: Aug. 25, 2005

(54) Mammalian pluripotent neural cells and uses thereof

(75) Inventors: Piotr Topilko, Antony (FR); Patrick Charnay, Bourg-La-Reine (FR); Geraldine Maro, Paris (FR)

Correspondence Address: NIXON & VANDERHYE, PC, 901 NORTH GLEBE ROAD, 11TH FLOOR, ARLINGTON, VA, 22203, US

(73) Assignee: INSERM, Paris cedex (FR)

(21) Appl. No.: 10/785,406

(22) Filed: Feb. 25, 2004

(51) Int. Cl. A61K 48/00; C12N 5/08

(52) U.S. Cl. 424/93.21; 435/368

(57) ABSTRACT

The present invention discloses the identification and characterization of a novel mammalian pluripotent neural cell population. More particularly, the present invention relates to compositions and methods allowing the identification, monitoring, culture and differentiation of boundary cap cells and their progeny. The invention also relates to methods of screening compounds that alter the growth, migration and/or differentiation of these cells, as well as to methods of producing functional cells in vitro, ex vivo or in vivo using said pluripotent cells. The invention can be used to detect, diagnose, monitor and/or treat various pathological conditions in mammalian subjects, including nervous diseases such as neurodegenerative condition, demyelination, pain, etc. The invention can be used for tissue engineering, to produce various differentiated cell types by differentiation of said pluripotent cells under appropriate conditions. The invention may also be used to identify genes or proteins that contribute to the nervous system function and/or integrity.

The present invention discloses the identification and characterization of a novel mammalian pluripotent neural cell population. More particularly, the present invention relates to compositions and methods allowing the identification, monitoring, culture and differentiation of boundary cap cells and their progeny. The invention also relates to methods of screening compounds that alter the growth, migration and/or differentiation of these cells, as well as to methods of producing functional cells in vitro, ex vivo or in vivo using said pluripotent cells. The invention can be used to detect, diagnose, monitor and/or treat various pathological conditions in mammalian subjects, including nervous diseases such as neurodegenerative condition, demyelination, pain, etc. The invention can be used for tissue engineering, to produce various differentiated cell types by differentiation of said pluripotent cells under appropriate conditions. The invention may also be used to identify genes or proteins that contribute to the nervous system function and/or integrity.

### BACKGROUND

The neural crest is a transient cell population that delaminates from the dorsal neural tube around the time of its closure and migrates to various sites in the vertebrate embryo. It gives rise to multiple cell types, including most neuronal and all glial components of the peripheral nervous system (PNS)¹. Neural crest cells fated to participate in the formation of dorsal root ganglia (DRG) migrate through the rostral half of each somite and condense in repetitive units on both sides of the spinal cord. Primary sensory DRG neurons subsequently develop in distinct but overlapping phases, according to their size and sensory modalities. Large diameter neurons that mediate transmission of proprioceptive and mechanoceptive information are born first, typically between embryonic day (E) 9.5 and 11.5 in the mouse, whilst small diameter nociceptive neurons involved in pain transmission appear later, between E10.5 and E13.5. Satellite cells, the glial cell-type associated with neuronal cell bodies in the DRG, are thought to develop also during this later period (for reviews, see ref. 1, 2, 3).

The neural crest also yields the axon-ensheathing cells of the PNS, Schwann cells. In this lineage, neural crest cells first give rise to precursors, then to immature Schwann cells, that differentiate into either myelinating or non-myelinating cells. It is still not established whether satellite cells and Schwann cells derive from independent or common precursors committed to a glial fate. Nevertheless two lines of evidence are consistent with the latter. First, clonal analysis of neural crest cultures identified bipotential precursors that give rise to at least two types of glia (Schwann-like cells, positive for the Schwann cell myelin protein (SMP) and satellite-like cells, SMP-negative)⁴. Second, recent studies have demonstrated that satellite cells can acquire Schwann cell properties in vitro^{5,6}. Besides satellite and Schwann cells, another PNS cell type, boundary cap (BC) cells, was shown to originate from the neural crest⁷. Altman and Bayer initially described BC cells on the basis of their close association with cranial and spinal nerve roots. They appear transiently in small clusters at the surface of the spinal cord, at prospective motor exit points (MEP) and dorsal root entry zone (DREZ), shortly before the arrival of axons (E11 in the rat)^{8,9} and disappear soon after birth in rat^{9,10}. Until recently BC cells have attracted limited attention and in the mouse only one BC-cell specific marker, Krox20, a gene encoding a zinc finger transcription factor, is available^{11,12}. Krox20 expression is initiated around E10 in BC cells associated with ventral and dorsal nerve roots and is maintained until E15.5 at least. From this stage onwards, Krox20 begins to be induced in immature Schwann cells throughout peripheral nerves¹³, preventing the subsequent identification of BC cells. In contrast, Krox20 is never expressed in satellite cells¹³. There is therefore a window, between E10 and E15.5, when Krox20 expression is restricted to BC cells in the trunk.

In vitro analyses performed on DRG neurons grown on spinal cord/dorsal root cryosections have suggested that BC cells might regulate the targeting of DRG axons to prospective entry sites in the spinal cord¹⁰. Also, ablation experiments have recently suggested a role for MEP BC cells in the confinement of motor neuron cell bodies within spinal cord motor columns¹⁴. However, inactivation of Krox20 in the mouse does not appear to affect the appearance of BC cells, cell migration or axon guidance at MEP and DREZ sites (ref. 11 and our unpublished results).

Accordingly, little is known so far of BC cell function, and the fate of these cells and their progeny is unknown.

### SUMMARY OF THE INVENTION

The present invention, for the first time, elucidates the fate of Boundary Cap (BC) cells. Using various genetic labelling techniques, the inventors have surprisingly shown that BC cell progeny gives rise to the Schwann cells on proximal ventral and dorsal roots. Furthermore, the inventors have demonstrated that BC-derived cells migrate along the nerve roots into the DRG where they differentiate into both glial satellite cells and nociceptive neurons. The present invention shows that the BC cells give rise to a broad range of cell types in the somatic peripheral nervous system, including Schwann cell precursors and possibly Schwann cells, satellite cells and both nociceptive and proprioceptive DRG neurons. BC cells therefore provide a secondary source of precursors to complete construction of the PNS after neural crest migration has ceased, and represent a novel pluripotent neural cell population suitable for therapeutic approaches, tissue engineering process, research and screening activities, particularly in the area of neurological disorders, including degenerative diseases, pain, trauma, and the like. The present invention also discloses methods and tools that allow the identification, characterization, purification and culture of said BC cells and/or their progeny or derivatives, thereby allowing a proper use of the therapeutic potential of these cells.

Accordingly, a first aspect of this invention resides in isolated mammalian BC cells, particularly isolated and expanded mammalian BC cells or their progeny or derivatives. One specific embodiment of the present invention is an isolated human BC cell, including a culture of human BC cell and expanded human BC cells, their progeny or derivatives.

Another aspect of this invention resides in a genetically modified mammalian BC cells or their progeny or derivatives. The genetically modified BC cell may comprise any heterologous nucleic acid molecule, either extra-chromosomal or integrated in the genome of said cell. The heterologous nucleic acid may confer on the BC cell, its progeny or derivative, any desired property, including the expression of a marker or reporter, the expression of a biologically active molecule, etc.

Another aspect of this invention resides in a method of producing neural pluripotent cells, comprising the ex vivo or in vitro culture, under suitable conditions, of mammalian BC cells or their progeny. The cells may be cultured under conditions allowing their survival and, optionally, their expansion (and/or amplification), preferably without loosing their pluripotent character.

Another aspect of this invention lies in a method of producing differentiated cells, comprising the ex vivo or in vitro culture of mammalian BC cells or their progeny, under conditions suitable for differentiation of said cells. Such conditions include the presence of appropriate factors, including trophic factors, stem cell factors, colony-stimulating factors, lymphokines, etc. Such conditions also include the presence of serum, nutrients, etc. Differentiation into distinct cell types can be monitored or detected using specific makers.

A further aspect of this invention resides in a pharmaceutical composition comprising BC cells, their progeny or derivatives. The pharmaceutical composition may further comprise any suitable vehicle, excipient or carrier, such as a saline solution, a buffer, etc. Preferred pharmaceutical compositions typically comprise from 10² to 10⁶ cells or more. The compositions may be packaged into any appropriate device, including a seringe, tube, pouch, ampoule, etc.

Another aspect of this invention lies in a method of tissue re-engineering, comprising administering to a subject in need thereof a suitable amount of mammalian BC cells or their progeny, under conditions suitable for differentiation of said cells in said subject, thereby allowing tissue re-engineering. Such conditions include the administration into proper sites, such as in, at or near a site of neurological disorder, where the organism produces, under physiological conditions, the appropriate environment for said BC differentiation and/or migration.

Another aspect of this invention lies in a method of reconstituting neural tissue in a subject, comprising administering to a subject in need thereof a suitable amount of mammalian BC cells or their progeny, under conditions suitable for differentiation of said cells into differentiated neural cells in said subject, thereby allowing tissue reconstitution. Such conditions include the administration into proper sites, such as in, at or near a site of neurological disorder, where the organism produces, under physiological conditions, the appropriate environment for said BC differentiation and/or migration.

The invention also resides in a method of screening compounds that modulate neuronal cell migration and/or differentiation, the method comprising contacting a candidate compound with a BC cell or a progeny thereof, and determining whether said compound modulates migration and/or differentiation of said cells. Contacting may be performed in vitro, ex vivo or in vivo. Determination of cell migration or differentiation can be carried out using various cell-specific markers, as described further in this application. The effect of the candidate compound on said cells may be compared to untreated cells or to cells treated in the presence of a reference compound.

A further aspect of this invention is a method of selecting nucleic acids or polypeptides that are specific for neural cell differentiation or migration, the method comprising preparing a nucleic acid sample from a test BC cell culture, contacting said sample with a reference nucleic acid sample under conditions allowing hybridisation to occur between complementary nucleic acids, said reference nucleic acid sample being obtained from a distinct cell type or from a differentiated or differentiating BC, and selecting one or several nucleic acids that are specific for the test BC cell culture. Such selected nucleic acids may be sequenced, cloned and/or expressed, to produce polypeptides that are specific for neural cell differentiation or migration. Such nucleic acids or polypeptides represent valuable targets for the design of therapeutic approaches.

The invention may be used in various mammals, including human subjects, to treat or prevent various pathological conditions, including more preferably neurological disorders.

### LEGEND TO THE FIGURES

FIG. 1. Comparison of reporter expression patterns along the nerve roots at E12.5. (A) Embryo section in situ hybridized with a Krox20 probe. (B,C) Single section from a Krox20^{GFP(D)/lacZ} embryo stained for β-galactosidase activity (B) and immunolabelled with an anti-GFP antibody (C). GFP is detected within the DRG and along the entire dorsal (arrow) and ventral (arrowhead) roots, whereas β-galactosidase is present in the BCs and the proximal parts of the roots and Krox20 mRNA restricted to the PNS/CNS interface. (D-G) Single section from a Krox20^{GFP(DT)/lacZ} embryo triple-immunolabelled with antibodies directed against β-III-tubulin (Tuj1, green) to reveal neurons and axons, β-galactosidase (red) and GFP (blue). BC cells and the proximal cells on the dorsal root express both β-galactosidase (E,G, arrows) and GFP (F,G, arrows). In contrast, β-galactosidase is not detected in distal GFP-positive cells (E-G, arrowheads). (E-G) are higher magnifications of D.

FIG. 2. Fate tracing of BC cells. (A,B) Transverse sections of E13.5 Krox20^{lacZ/+} and Krox20^{OCre/+},R26R embryos, stained by X-gal. In the Krox20^{lacZ/+} embryo (A), lacZ-expressing cells are located at the boundary cap (BC) and along the proximal part of the root (arrow). In contrast, in the Krox20^{Cre/+},R26R embryo (B), in which the progeny of Krox20-expressing cells is labelled, positive cells are found along the entire root and within the DRG. (C-F) Krox20^{Cre/+},R26R embryo sections immunostained with antibodies directed against β-galactosidase (red) and β-III-tubulin (Tuj1, blue), and counterstained with a nuclear marker (green) at the indicated stages. (C) At E10.75, β-galactosidase-positive cells are located directly adjacent or very near to the spinal cord (SC), reflecting Krox20 expression at the BC. (D,F) At E11.25, more numerous cells are labelled and cover the dorsal and ventral roots. (E) At E11.75, β-galactosidase-positive cells have reached the DRG. The arrowheads indicate the most ventral β-galactosidase-positive cells in connexion with the dorsal root.

FIG. 3. Dorsal root glial cells are derived from the BC. (A-D) E12.5 Krox20^{Cre/+},R26R embryo sections were labelled with antibodies directed against β-galactosidase (red) and β-III-tubulin (Tuj1, blue), and a nuclear marker (green). (A-C) Dorsal roots are covered by lacZ-expressing cells, which extend cytoplasmic processes (arrows) preventing contacts between axons and neighbouring perineurial cells (arrowhead). (D) Transverse section through the dorsal root showing lacZ-expressing cells wrapping bundles of axons. Perineurial cells (stars) do not express the reporter gene. (E,F) E12.5 Krox20^{Cre/+},R26R embryo section labelled with antibodies against β-galactosidase (red) and ErbB3 (green). This double labelling confirms the glial identity of lacZ-positive cells on the dorsal root.

FIG. 4. Analysis of BC-derived cells in embryonic DRGs. (A) Transverse section of a Krox20^{Cre/+},R26R E13.5 embryo showing that X-gal-positive cells are present in the DRG and mostly located in its medial part. (B) Krox20^{Cre/+},R26R E12.5 DRG section labelled with antibodies directed against β-galactosidase (red) and β-III-tubulin (Tuj1, blue). Some of the β-galactosidase-positive cells are neurons (arrows), whereas others are negative for the neuronal marker (arrowheads). (C) Same section as (B) showing Tuj1 staining in grey. (D,E) Krox20^{Cre/+},R26R E13.5 DRG section labelled with antibodies against β-galactosidase (red), β-III-tubulin (Tuj1, green) and TrkA (blue), a receptor for neurotrophins expressed in developing small-diameter sensory neurons. Virtually all β-galactosidase-positive neurons (Tuj1-positive, arrows) also express TrkA. Arrowheads point to glial and/or undifferentiated (Tuj1-negative) β-galactosidase-positive precursors. Scale bars: 50 μm.

FIG. 5. Analysis of BC-derived cells in the adult DRG. (A) DRGs from Krox20^{lacZ/+} (left) and Krox20^{Cre/+},R26R (right) mice stained by X-gal. Whereas extensions of β-galactosidase-positive Schwann cells are observed in both cases, only the Krox20^{Cre/+},R26R DRG contains scattered, X-gal-positive cell bodies (note that lacZ is expressed at higher levels from the Krox20 locus than from the ROSA locus). (B) Semi-thin section through a Krox20^{Cre/+},R26R DRG stained for β-galactosidase activity by Bluo-Gal. β-galactosidase is observed mostly in small-to-medium diameter neurons. (C,D) Ultra-thin section through the same ganglion, showing the Bluo-Gal precipitate in a neuron (C) and in a presumptive satellite cell associated with a neuron (D). (E,F) Krox20^{Cre/+},R26R DRG sections labelled with antibodies against β-galactosidase (red) and the pan-neuronal marker NeuN (E, green) or the glial marker GFAP (F, green). β-galactosidase-positive cells express either neuronal (E, arrowheads) or glial (F, arrowhead) markers. In (F), a β-galactosidase-positive presumptive neuron (star) is located near the 13-galactosidase-positive satellite cell cluster. Scale bars: 5 μm (C,D), 30 μm (E,F).

FIG. 6. Characterisation of the subtype of BC-derived sensory neurons in the adult DRG. (A,B) Krox20^{Cre/+},R26R DRG section double labelled with an antibody against β-galactosidase (red) and the IB4 lectin (green), which marks a subpopulation of nociceptive neurons. IB4-positive (A) and -negative (B) β-galactosidase-positive cells are shown as examples. (C,D) Krox20^{Cre/+},R26R DRG section labelled with antibodies against β-galactosidase (red) and the calcitonin-gene related peptide (CGRP, green), which marks peptidergic nociceptors. CGRP-positive (C) and -negative (D) β-galactosidase-positive cells are shown as examples. (E,F) Krox20^{Cre/+},R26R DRG section labelled with antibodies against β-galactosidase (red) and parvalbumin (PV, green), which marks a subpopulation of proprioceptive neurons. Note the cell positive for both markers in E (arrowhead). Interestingly, β-galactosidase-positive satellite cells are mostly found associated with PV-positive neurons (stars in F). Scale bars: 15 μm (A-D), 30 μm (E,F).

FIG. 7. Krox20 expressing cells are detected at peripheral nerve entry points in the human fetus. Semi-thin sections of a human fetus (7-8 weeks of gestation) were immunolabeled with an anti-Krox20 antibody. Labeled cells (arrows) are found within clusters of cells located at peripheral nerve entry points (hatched line). Thus, these cells have a morphology and position consistent with a BC cell identity, and express a marker specific for BC cells in the mouse embryo.

### DETAILED DESCRIPTION OF THE INVENTION

Boundary Cap Cells and their Production

Boundary cap (BC) cells are neural crest derivatives that form clusters at the surface of the neural tube at the level of both entry and exit points of peripheral nerve roots. During early stages, these cells express a specific marker, Krox-20 (or its human homologue), a gene encoding a zinc-finger transcription factor. Krox-20-specific reagents (e.g., probes, antibodies, aptamers, ligands, and the like) can be used to detect and characterize BC in certain tissues, including neural crest. Boundary cap (BC) cells may also be obtained or isolated from somatic adult tissues, such as for instance in dorsal root ganglia. BC cells represent pluripotent neural cells and usually do not express markers that are specific for differentiated cell populations, such as β-III-tubulin, NeuN, TrkA. Such markers become expressed at the surface of or in BC cells derivatives, i.e., cells that have differentiated into mature cells from BC cells. BC cells may also be enriched based on the expression of the Monoamine oxydase B gene, which is expressed in BC cells and in glial precursors on the dorsal root⁴⁰.

BC progeny designates cells produced by in vitro or ex vivo culture of BC cells previously isolated from a biological sample. BC cells' progeny retain the pluripotent phenotype of BC cells.

A particular object of the present invention relates to a method of producing neural pluripotent cells, the method comprising the ex vivo or in vitro culture, under suitable conditions, of mammalian Boundary Cap cells. An other object of this invention is a method of producing differentiated cells, the method comprising the ex vivo or in vitro culture of mammalian Boundary Cap cells or their progeny, under conditions suitable for differentiation of said cells. The BC cells may be derived from various sources, such as the neural crest, the dorsal ganglia, etc. They may be obtained by enzymatic or mechanic treatment of the tissue (e.g., pipette disruption, trypsin digestion, collagenase digestion, etc.), followed by culture under appropriate conditions. They are preferably autologous with respect to a subject. In that case, a particular method of this invention comprises (i) providing a biological sample from a subject, wherein said sample comprises BC cells and (ii) culturing and/or differentiating said cells in vitro or ex vivo. The BC cells may be cultured in the presence of differentiation factors selected from trophic factors, stem cell factors, colony-stimulating factors and lymphokines. Differentiation into distinct cell types can be detected using cell-specific makers, as listed below.

| Marker | Cell Type |
|---|---|
| β-III tubulin | neurons |
| NeuN | neurons |
| NGF receptor, TrkA | neurons, particularly |
| | nociceptive neurons |
| GFAP | glial cells |
| Neuregulin receptor, ErbB3 | glial cells |
| Calcitonin-Gene Related Peptide | nociceptive neurons |
| (CGRP) | |
| Isolectin B4 (IB4) | nociceptive neurons |
| parvalbumin | proprioceptive neurons |

A particular embodiment of this invention resides in a method of producing neurons in vitro or ex vivo, the method comprising culturing mammalian BC cells or their progeny under conditions allowing differentiation of said cells, and selecting or recovering cells that express neuron-specific markers, preferably selected from β-III tubulin, NeuN, NGF receptor TrkA, CGRP and IB4.

An other specific embodiment of this invention concerns a method of producing nociceptive neurons in vitro or ex vivo, the method comprising culturing mammalian BC cells or their progeny under conditions allowing differentiation of said cells, and selecting or recovering cells that express nociceptive neuron-specific markers, preferably selected from CGRP and IB4.

A further specific embodiment of this invention is a method of producing schwann cell precursors in vitro or ex vivo, the method comprising culturing mammalian BC cells or their progeny under conditions allowing differentiation of said cells, and selecting or recovering cells that express schwann cell precursors-specific markers, preferably selected from GFAP.

The above methods may be performed into any appropriate device, including a tube, plate, flask, pouch, etc. Upon culture and/or differentiation, the cells may be formulated into any appropriate diluent or excipient, or they may be stored (e.g., to produce cell banks). In particular, the cells may be placed in suspension in any desired medium and used as such, as a therapeutic product. Indeed, implantation of BC cells, their progeny or derivatives, in suspensions, notably autologous cells, is particularly suitable for clinical applications in the regeneration of nervous tissue. To that effect, the cells are preferably washed, several times, in a fresh culture medium, and then exposed to any physiological solution (buffer, saline, etc.) that is adapted for therapeutic use, notably for administration in vivo. Typically, the cells can be exposed to a 1-5% albumin solution or to an autologous serum, in a volume that is selected to obtain the desired cell concentration. For example, immersion of cells in a volume of less than 0.5 ml enables a concentration of autologous cells to be obtained in excess of 10⁵ cells/ml, for example. The cells can be placed in tubes, ampoules, syringes, etc., under sterile conditions, and can be utilized for injection. The cells may also be placed in a gelified medium or substrate, to facilitate their subsequent implantation.

In this regard, a particular aspect of this invention is a pharmaceutical composition comprising BC cells, their progeny or derivatives thereof (i.e., differentiated cells derived from BC or their progeny), and a suitable vehicle, excipient or carrier. The pharmaceutical composition typically comprises from 10² to 10⁶ cells or more. The composition may further comprise any suitable stabilizing agent, protein, fluid, isotonic solution, buffer, saline solution, etc., that is compatible for pharmaceutical use, preferably in human subjects.

An other particular aspect of this invention resides in a cell bank, wherein said cell bank comprises a plurality of compartments, wherein said compartments comprise BC cells, their progeny or derivatives, derived from different patients. The cells may be stored under various conditions, including lyophilised or frozen.

Boundary Cap Cells Represent Pluripotent Neural Cells

In the present work, the role and fate of BC cells have been investigated using several Krox20 knock-in mouse lines. In particular, we took advantage of a knock-in of the Cre recombinase gene in the Krox20 locus^{15,16} that allowed us to follow the fate of BC cell progeny, using a lacZ transgene which is permanently activated by Cre-mediated recombination¹⁷. We demonstrate that BC cell progeny leave the DREZ and MEP and migrate along peripheral axons to rapidly colonize dorsal and ventral spinal nerve roots and DRGs. At E12.5 all Schwann cell precursors located along the dorsal roots are derived from BC cells. In the adult DRG, BC cell derivatives include both satellite cells and nociceptive neurons. These data indicate that BC cells constitute a late-surviving reservoir of pluripotential neural cell precursors in the PNS.

BC Contribution to the PNS

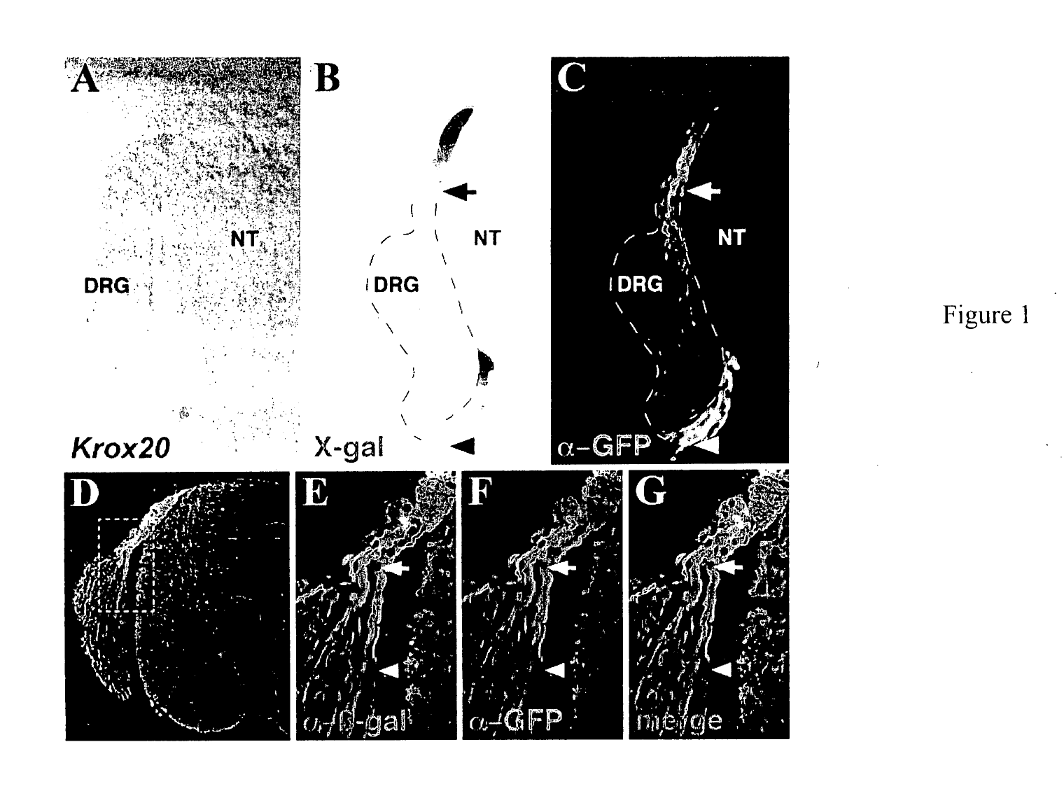

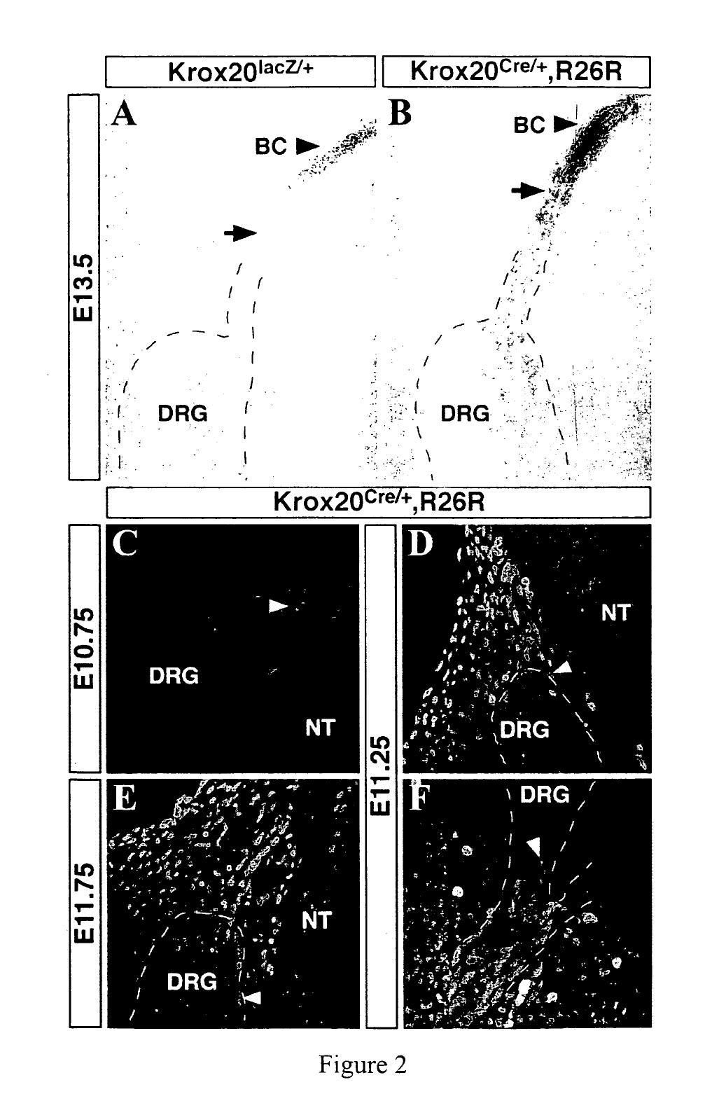

Here we show by using both transient and permanent labelling of BC cells that their progeny leave the DREZ and the MEP and migrate along attached spinal nerve roots. Whilst formation of BC cell clusters is accompanied by activation of the Krox20 gene, cells lose Krox20 expression upon emigration as indicated by following different markers (Krox20 mRNA, knock-in of lacZ or of GFP, FIG. 1). Time course analysis indicated that emigration is initiated around E11 (FIGS. 2C and 2D). It could therefore occur as soon as BC cells have settled at the DREZ and MEP and peripheral axons become available as a substrate for traction.

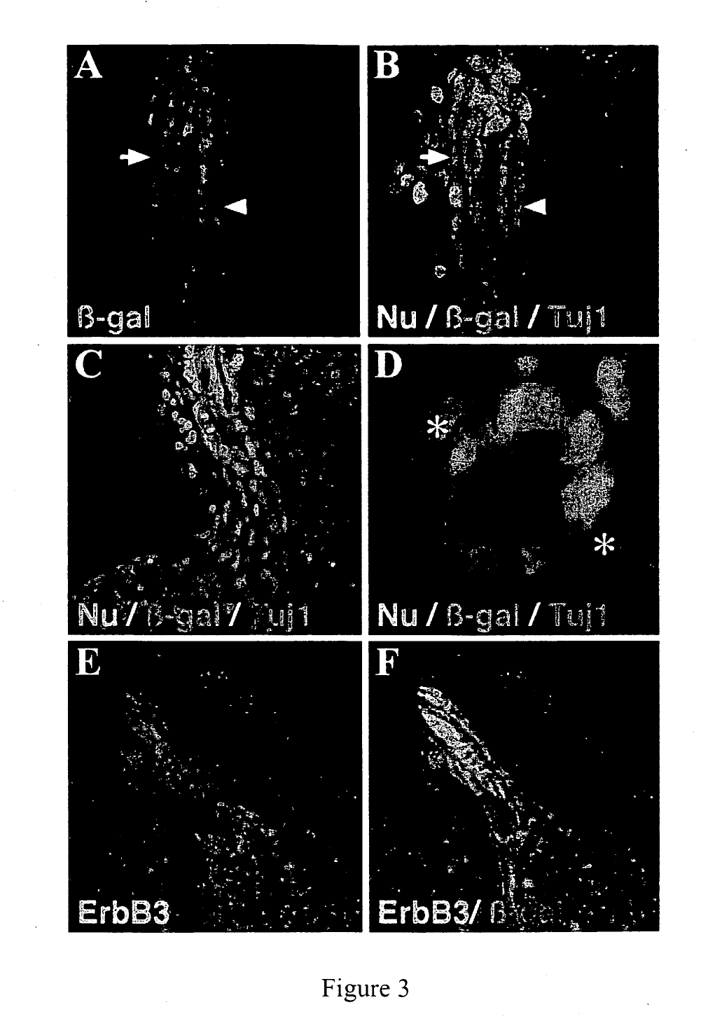

Emigrating BC-derived cells first encounter proximal portions of dorsal or ventral nerve roots. We show that by E12.5 BC cell derivatives completely ensheath dorsal root axons (FIG. 3). If emigration continues beyond this stage, it is possible that cells stay only transiently on spinal nerve roots and are continuously replaced by novel immigrants. Alternatively, the early immigrants may settle on the nerve roots whereas the late ones might continue to migrate distally. Those derivatives that settle on the roots are Schwann cell precursors, based on their morphology, their intimate ensheathment of axons and expression of the PNS glial cell marker ErbB3. With the activation of Krox20 expression at around E15.5 in Schwann cell precursors along the entire nerves however, the fate of BC-derived cells could not be followed beyond this stage. Thus it could not be unambiguously established whether all spinal nerve root Schwann cells are of BC-cell origin. Similarly, it also precludes the investigation of the contribution of the BC cells to Schwann cells located in DRG and peripheral nerves. Addressing these issues will require the development of a means of labelling BC cell progeny with a marker not expressed in Schwann cells arising from the mainstream neural crest.

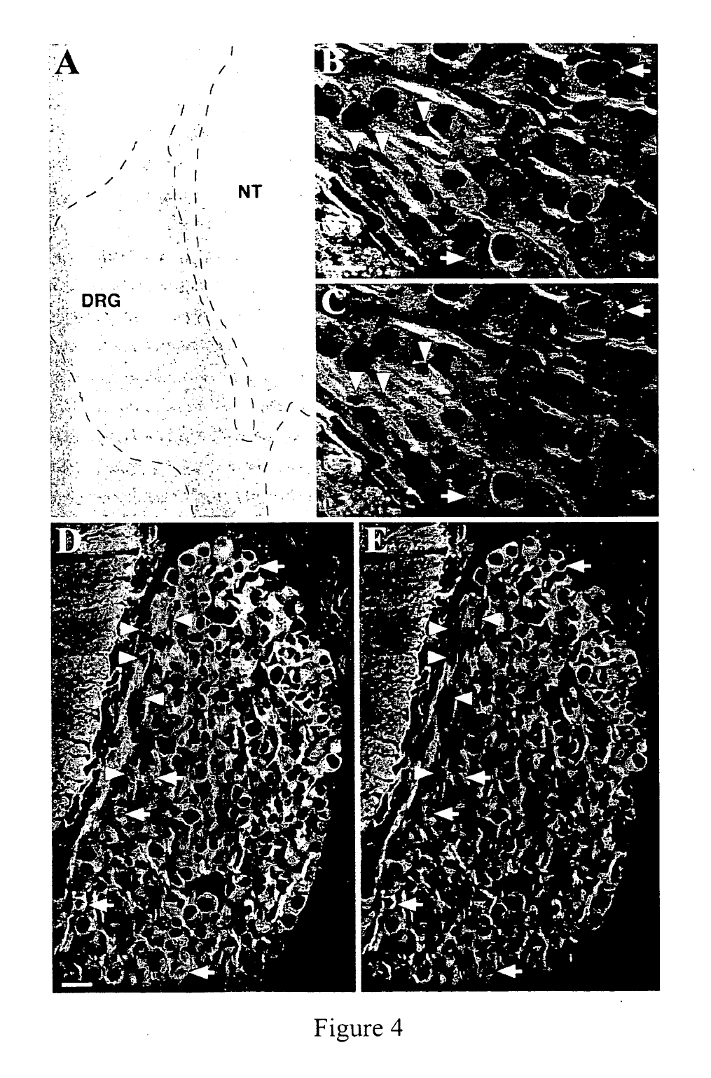

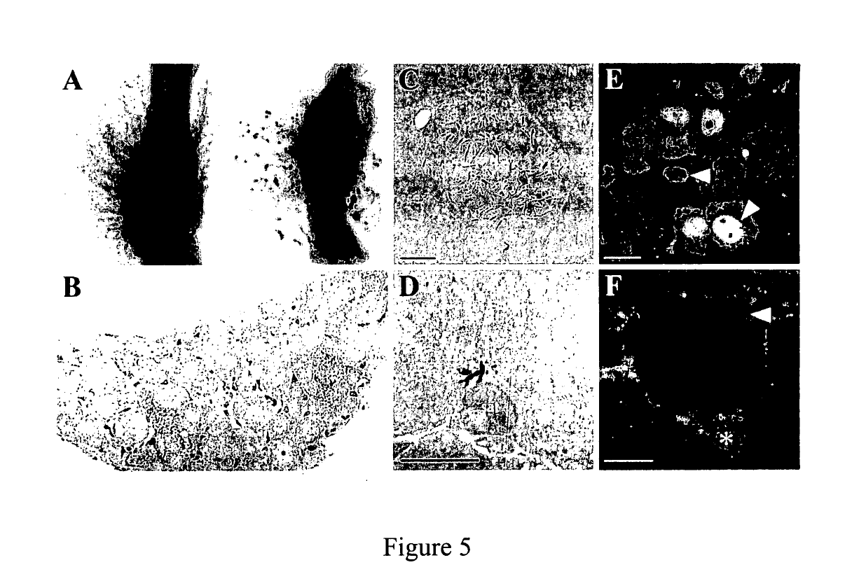

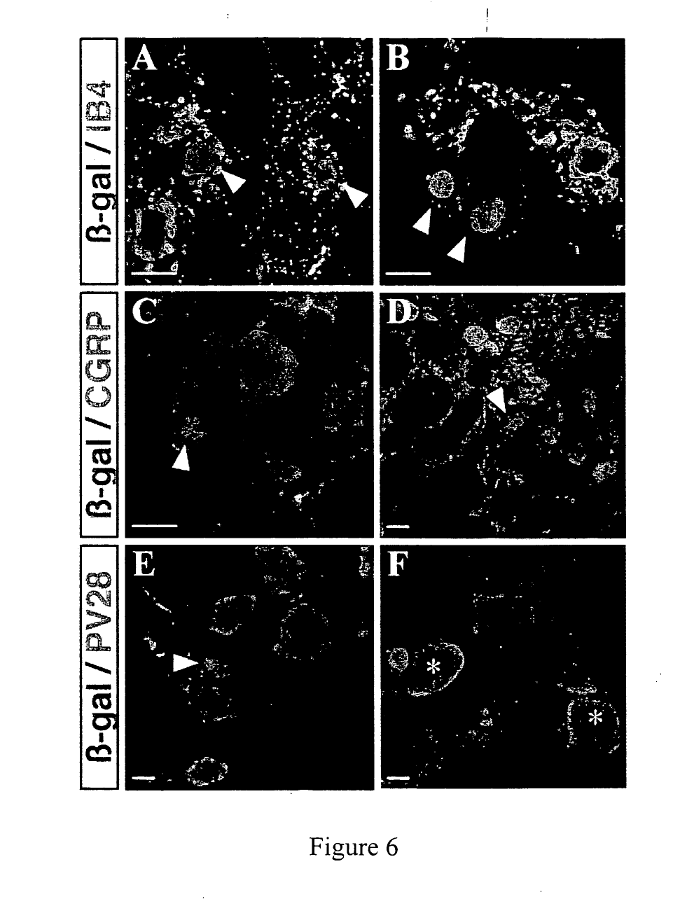

BC-derived cells first reach the DRGs as early as E11.5 (FIG. 2D-F) and significant colonisation of the DRG continues at least until E12.5 (FIG. 1C), (FIG. 4A). Unlike in Schwann cells, Krox20 is never expressed in DRG neurons and satellite cells¹³, providing us with the opportunity to trace the fate of BC-derived cells in the DRGs up to adulthood. From E12.5 onwards, part of the BC cell progeny in the DRGs was positive for the neuronal marker β-III-tubulin (FIGS. 4B and 4C). In the adult, a combination of morphological and immunolabelling analyses showed that approximately 5% of the DRG neurons are derived from Krox20-expressing cells (FIGS. 5 and 6). Although we cannot exclude the possibility that Schwann cells, that express Krox20 from 15.5 onwards and therefore are labelled by our fate tracing methods beyond this stage, might contribute to this population, our data strongly suggests that these neurons originate from BC cell immigrants. Virtually all BC-derived DRG neurons are of small-to-medium diameter size range and at least 87% of them express nociceptive-specific markers (FIG. 6), whereas only 70% of DRG neurons are nociceptive. Consistent with these findings, only 1% of BC-derived DRG neurons expressed the proprioceptive afferent-specific marker parvalbumin as compared to 15% of the total DRG neuron population^{25,28}. Together these data suggest that BC-derived cells are biased towards a DRG nociceptive afferent fate. A recent screen for genes specifically expressed in sensory neurons revealed substantial molecular diversity amongst nociceptors²⁹. Further studies will therefore be required to determine whether BC cells give rise to particular subtypes of nociceptive neurons. In addition to neurons, we found that BC-derived cells give rise to non-neuronal cells in the DRGs (FIG. 5). Characterisation of these cells in the adult DRG indicated that most are satellite cells, based on their morphology and the expression of GFAP (FIG. 5E).

Altogether our data indicate that the BC cells give rise to a broad range of cell types in the somatic peripheral nervous system: Schwann cell precursors and possibly Schwann cells, satellite cells and both nociceptive and proprioceptive DRG neurons. BC cells therefore provide a secondary source of precursors to complete construction of the PNS after neural crest migration has ceased.

Novel Insights into Neuronal and Glial Cell Differentiation in the DRG

Birth-dating studies in mouse DRG have shown that neurogenesis occurs in two successive, but overlapping waves¹⁹. Large-diameter neurons are born first, between E9.5 and E11.5, whereas small-diameter neurons are generated later, between E10.5 and E13.5. Glial satellite cells are thought to differentiate also during this latter period^{2,19}. Transcription factors of the bHLH family, neurogenin-1 and -2, have been shown to be required for these two waves of differentiation^{30,31}, but it remains unclear how the relative proportions of the two classes of neurons found in the adult is established. Moreover, it is not known whether the choice is intrinsic (each neuronal population arises from a specific committed progenitor pool) or extrinsic (the progenitors are of a unique type and their fate is determined by signals from their environment and adjacent cells during their differentiation). In the case of BC-derived cells, we do not know whether their differentiation within the DRGs is governed by the same mechanisms as those acting on cells derived from the major neural crest migration. However, their fate is consistent with such a possibility: most BC-derived cells enter the DRG after E11.5, at a time when large-diameter neuron differentiation is almost complete, and very few BC-derived cells differentiate into large-size proprioceptive neurons, whereas most of them yield small-diameter nociceptive neurons. The availability of the markers described in this work and specific for BC cells or BC-derived cells now offers the possibility of purifying these cells and directly testing factors that control sensory neuron differentiation in the DRGs. It will be of particular interest to perform transplantations of mouse BC cells into early chick DRGs to distinguish between extrinsic and intrinsic determination programs and to attempt to alter the expression of BC cell genes that have been implicated in the control of their differentiation.

Concerning the choice between neuronal and glial fates in the DRG, available evidence suggests the existence of an extrinsic mechanism³². Hence, the activation of the Notch pathway by differentiated DRG neurons is thought to induce a glial fate in adjacent progenitors^{33,34}. Our observations of BC-derived cells do not address this issue, but provide additional information on the behaviour of glial precursors. We noticed that BC-derived satellite cells were usually found in clusters surrounding a single neuron and were preferentially associated with large-diameter parvalbumin-positive neurons (FIG. 6F). This suggests a mechanism whereby once an undetermined progenitor has been induced by a neuron to adopt a satellite glial fate, it will stay in association with this neuron and continue to proliferate in order to generate clonally related satellite cells. Furthermore, the bias in favour of BC-derived glia associating with large-diameter neurons, which comprise the majority of the neurons in the DRG at the time of arrival of BC-derivatives, suggests that a permanent association between the neuron and uncommitted progenitor occurs very early, as soon as both cell types are born. Here again, the possibility of manipulating BC cells as indicated above should allow us to further test these possibilities and reveal the basis of glial versus neuronal fate selection.

It has thus been established that BC cells can give rise to various PNS cell types, including Schwann cell precursors, satellite cells, proprioceptive and nociceptive neurons. BC cells could furthermore constitute a late-surviving stem cell population. Indeed, our data show that BC cell progeny can populate the spinal nerve roots and give rise to both satellite glial cells and a sub-population of neurons in the DRG. Furthermore, our data indicate that glial precursors derived from BC cells continue to proliferate within the DRG and establish a permanent relation with a specific neuron. The contribution of BC cell derivatives should thus be very substantial in pathological situations, particularly those characterized by the loss of PNS cells, and should constitute a reservoir of pluripotent neural cells for the PNS. It is thus proposed that BC cells contribute some of the late cohort of proprioceptive neurons. Similarly, it is proposed to use BC cells or derivatives thereof in situations of peripheral nerve demyelination, injury and regeneration.

Use of Boundary Cap Cells in Cell Therapy and Tissue Reconstitution

Based on the above evidence regarding BC functions and fate, and the above description of suitable methods of producing, culturing, expanding and differentiating said cells, the present invention now allows, for the first time to use BC cells in cell therapy and tissue reconstitution approaches, particularly in subject suffering from nervous diseases, more preferably peripheral nervous diseases, including pain, nervous system degeneration, trauma, injury, demyelination, and the like.

A general aspect of this invention is a method of treating a subject having a disease associated with nerve dysfunction, the method comprising administering to the subject an amount of BC cells, their progeny or derivatives, effective for said cells to treat said dysfunction. The cells are preferably autologous with respect to the patient, i.e., have been isolated from the patient shortly before treatment, or stored in a bank as described above.

Within the context of the present invention, the term “treating” designates the compete treatment of a disease as well as reducing or alleviating a condition, such as a pain, nerve degeneration, nerve demyelination, etc. Furthermore, the treatment may be performed either alone or in combination with other therapeutically active agents or conditions.

A particular embodiment of this invention resides in a method of tissue re-engineering, comprising administering to a subject in need thereof a suitable amount of mammalian BC cells or their progeny, under conditions allowing differentiation or migration of said cells in said subject, thereby allowing tissue re-engineering.

An other particular embodiment of this invention resides in a method for reconstituting neural tissue in a subject, comprising administering to a subject in need thereof a suitable amount of mammalian BC cells or their progeny, under conditions suitable for differentiation of said cells into differentiated neural cells in said subject, thereby allowing tissue reconstitution.

A further particular embodiment of this invention lies in a method of treating, reducing or alleviating pain in a subject, the method comprising administering to the subject an amount of mammalian BC cells or their progeny, under conditions allowing said cells to differentiate into nociceptive neurons in said subject.

An alternative method of treating, reducing or alleviating pain in a subject comprises culturing mammalian BC cells or their progeny in vitro or ex vivo under conditions allowing said cells to differentiate into nociceptive neurons, and administering to the subject said nociceptive neurons.

In the above methods, the mammalian BC cells are preferably autologous with respect to the subject. Furthermore, the cells may be genetically engineered to contain any selected nucleic acid molecule.

The cells may be administered in, at or near a site of neurological disorder. In particular, where the subject suffers from peripheral nerve demyelination, injury or degeneration, the cells can be administered to the patient at a site of demyelination, injury or degeneration. The cells may be injected using any appropriate device, such as a serynge for instance, or they may be injected during surgery.

Use of Boundary Cap Cells for Drug Screening

The invention also provides novel methods of screening biologically active drugs, which are based on the effect of such drugs on BC activity.

A particular object of this invention thus resides in a method of screening compounds that modulate neuronal cell migration and/or differentiation, the method comprising contacting a candidate compound with a BC cell or a progeny thereof, and determining whether said compound modulates migration and/or differentiation of said cells.

In a particular embodiment, the contacting is performed in vitro or ex vivo. Such an embodiment may be performed in any suitable device, such as plates, tubes, dishes, flasks, etc. Typically, the assay is performed in multi-wells plates. Several test compounds can be assayed in parallel. Furthermore, the test compound may be of various origin, nature and composition.

In an other embodiment, the contacting is performed in vivo, e.g., in a non-human animal or a test organism. In this embodiment, a candidate compound is directly injected to the test organism containing BC cells, and the fate of said cells upon injection is assessed. To facilitate such an assessment, the BC cells may be labelled, e.g., using genetic markers, reporter constructs, etc.

Differentiation of the BC cells into various cell types can be monitored or determined using particular cell markers, as mentioned before.

In this respect, a further aspect of this invention resides in a method of screening compounds that modulate neuron cell differentiation, the method comprising contacting a candidate compound with a BC cell or a progeny thereof, and determining whether said compound modulates differentiation of said cells into cells expressing a neuronal marker, preferably selected from the group of β-III tubulin, NeuN, NGF receptor TrkA and parvalbumin.

A further aspect of this invention resides in a method of screening compounds that modulate glial cell differentiation, the method comprising contacting a candidate compound with a BC cell or a progeny thereof, and determining whether said compound modulates differentiation of said cells into cells expressing a glial cell marker, preferably selected from the group of GFAP and ErbB3.

A further aspect of this invention resides in a method of screening compounds that modulate nociceptive neuron cell differentiation, the method comprising contacting a candidate compound with a BC cell or a progeny thereof, and determining whether said compound modulates differentiation of said cells into cells expressing a nociceptive cell marker, preferably selected from the group of NGF receptor TrkA, Calcitonin-Gene Related Peptide (CGRP) and Isolectin B4 (IB4).

A further aspect of this invention resides in a method of screening compounds that modulate proprioceptive neuron cell differentiation, the method comprising contacting a candidate compound with a BC cell or a progeny thereof, and determining whether said compound modulates differentiation of said cells into cells expressing a proprioceptive cell marker, preferably parvalbumin.

A further aspect of this invention resides in a method of screening compounds that modulate satellite cell differentiation, the method comprising contacting a candidate compound with a BC cell or a progeny thereof, and determining whether said compound modulates differentiation of said cells into cells expressing a satellite cell marker, preferably GFAP.

Further aspects of the present invention will be disclosed in the following experimental section, which should be considered as illustrative and not limiting the scope of the present application. All publications, applications or other documents cited in the present application are incorporated therein by reference.

### EXAMPLES

Methods

Mouse Lines

All the mouse lines used in this study were maintained in a mixed C57B16/DBA2 background. The Krox20^{lacZ} allele carries an in-frame insertion of lacZ coding sequence with the second exon of Krox20¹¹. In the Krox20^{Cre/+} allele, the Krox20 coding sequence was substituted by the Cre-recombinase coding sequence¹⁵. The Krox₂₀^{GFP(DT)} allele consists in an insertion of the GFP gene at the level of the Krox20 initiation codon¹⁴. The R26R transgenic line was kindly provided by P. Soriano¹⁷.

Detection of β-Galactosidase Activity and in Situ Hybridisation

Mouse embryos were dissected in PBS (phosphate buffered saline) and fixed in 4% paraformaldehyde (PFA) at room temperature for 20-40 min depending on the stage (day of plug is E0.5). Whole-mount β-galactosidase in situ detection was performed as described¹¹. Briefly, embryos were stained overnight at 30° C. in PBS containing 2 mM MgCl₂, 0.1% Triton-X100, 5 mM K₃Fe(CN)₆, 5 mM K₄Fe(CN)₆ and 0.4 mg/ml X-Gal. Embryos were then post-fixed in 4% PFA for 2 h at 4° C. and washed in PBS. Sections (60 μm) were cut on a freezing microtome after equilibration in 30% sucrose. In situ hybridisation with a Krox20 probe was performed on whole-mount neural tubes (with attached DRGs) as described previously³⁸ and 30 μm sections were subsequently cut on a cryostat.

Immunohistochemistry

For double-immunolabelling, embryos or DRGs were dissected, fixed in 4% PFA for 1-3 h at 4° C. prior to equilibration in 30% sucrose. They were then imbedded in Tissue-Tek (Sakura) and 16 μm sections were prepared on a cryostat. Non-specific binding sites were blocked with PBS containing 0.2% Triton X-100 and 10% goat serum. Primary antibodies were applied in the same solution overnight at 4° C. Fluorophore-conjugated secondary antibodies (Jackson Immuno Research; Silenus) were applied at 1/400 dilution in PBS containing 0.1% Triton-X100 for 2 h at room temperature. Sections were mounted in Vecta Shield (Vector). The primary antibodies were used at the following dilutions: rabbit anti-TrkA (gift of Dr. L. Reichardt, 1/2000), mouse anti-β-tubulin-type III (Tuj1, Babco, 1/1000), mouse anti-NeuN (Chemicon, 1/500), mouse anti-GFAP (Sigma, 1/150), mouse anti-CGRP (Chemicon, 1/500), rabbit anti-ErbB3 (Santa Cruz, 1/200), rabbit anti-PV28 (Swant, 1/1000), rabbit anti-β-galactosidase (Cappel, 1/700), goat anti-β-galactosidase (Biogenesis, 1/1000), rabbit anti-GFP (Molecular Probes, 1/500). For double-labelling with Griffonia simplicifolia IB4 lectin, sections were incubated with 12.5 μg/ml FITC-conjugated EB4 lectin (Sigma) together with the secondary antibody. Nuclei were counterstained with Sytox Green (Molecular Probes). Immunofluorescence pictures were acquired on a Leica TCS 4D confocal microscope and assembled using Adobe Photoshop.

Semi-Thin Sections and Electron Microscopy

Post-natal DRGs (P30) were isolated from animals perfused with 0.5% glutaraldehyde in phosphate buffer (PB, pH 7.4), fixed with 0.5% glutaraldehyde for 1 h at 4° C., washed in PB. Staining with Bluo-Gal (1 mg/ml, Sigma) was performed as described for X-Gal (see above), overnight at 30° C. Bluo-gal was used for electron microscopy because it produces a heavily electron-dense precipitate and is less diffusable than X-Gal³⁹. The following day the samples were post-fixed in 1.6% glutaraldehyde in PB for 1 h at 4° C., osmificated in 1% OsO₄ (Sigma) in PB for 1 h, dehydrated in ethanol (30%, 50%, 70%), stained with uranyl acetate in 70% ethanol for 1 h, dehydrated in ethanol (80%, 90%, 100%) and embedded in Durcupan (Fluka). 1 μm semi-thin sections were stained with toluidine blue. Ultra-thin sections were cut and stained with uranyl acetate and lead citrate. The sections were observed in Tecnai 12 (FEI, Phillips) electron microscope.

Results

BC Cell Progeny Migrate Along Nerve Roots and into the DRG

To investigate the emigration and dispersal of BC cell derivatives from their original location at the DREZ and MEP, we first compared the pattern of Krox20 mRNA, as revealed by in situ hybridisation, to reporter protein distributions in knock-in alleles into this locus. Whereas Krox20 mRNA is restricted to the BC cells, the greater stability of reporter proteins should make it possible to trace migrating BC cell progeny. We therefore analysed the patterns of β-galactosidase in embryos carrying a knock-in of lacZ (Krox20^{lacZ} allele¹¹), as revealed by X-gal staining or by immunohistochemistry, and of GFP in embryos carrying a knock-in of the GFP gene (Krox20^{GFP(DT)} allele¹⁴), as revealed by anti-GFP immunolabelling.

At E12.5, Krox20 mRNA localised to BC cells at the DREZ and MEP (FIG. 1A), as expected from previous work¹². In compound heterozygous Krox20^{lacZ/GFP(DT)} embryos, labelling of transverse sections revealed that BC cells were X-gal-positive and that the staining extended along the proximal half of both dorsal and ventral roots (FIG. 1B). Analysis of anti-GFP antibody staining on the same section revealed more widespread expression, covering the entire roots and extending into the DRG (FIG. 1C; note that GFP labelling is partially quenched by the X-gal precipitate). These differences in the extent of GFP and β-galactosidase labelling were also observed when β-galactosidase expression was revealed using two distinct antibodies (FIG. 1D-1G, and data not shown). Similar data were obtained for each marker in single heterozygous embryos (data not shown). These results are all consistent with extensive distal migration of BC cells along the spinal nerve roots and into the DRG.

To confirm and extend these results, we made use of another knock-in allele, Krox20^{Cre}, where Cre recombinase has been shown to faithfully recapitulate the Krox20 expression pattern¹⁵. Combination of this allele with the R26R reporter transgene, which leads to permanent lacZ activation upon Cre-mediated recombination¹⁷, allows the progeny of Krox20-expressing cells to be traced^{15,16} In Krox20^{lacZ/+} E13.5 embryos, as at E12.5, X-gal-positive cells were observed within BCs and in the proximal portions of dorsal nerve roots (FIG. 2A). In contrast, in Krox20^{Cre/+},R26R embryos, labelled cells were observed as a continuous stream extending from the BCs, along the entire dorsal root and into the DRG (FIG. 2B). These data are also consistent with the migration of DREZ BC-derived cells towards the DRG. The timing of BC-derived cell dispersal was studied between E10.5 and E12.5 in Krox20^{Cre/+},R26R embryos by β-galactosidase immunostaining. The earliest β-galactosidase-positive cells were detected at the DREZ in E10.75 embryos (FIG. 2C, arrowhead). At E11.25, labelled cells were observed to have extended along the entire length of the dorsal root but had not yet entered the DRG (FIG. 2D). Interestingly, in the ventral root β-galactosidase-positive cells were also observed up to the posterior surface of the DRG (FIG. 2F, arrowhead), raising the possibility that ventral BC-derived cells might also invade the DRG. At E11.75, a continuous stream of β-galactosidase-positive cells was observed extending from the DREZ BC to the centre of the DRG (FIG. 2E, arrowhead). This time-course is fully consistent with the progressive invasion of nerve roots and DRGs by BC-derived cells.

BCs Generate the Glial Component of Dorsal Roots

We next explored the fate of BC-derived cells migrating along the dorsal root by characterising their phenotypes. Analysis of sections of Krox20^{Cre/+},R26R embryo containing dorsal roots co-labelled for β-galactosidase, a nuclear marker and a neuronal marker (β-III-tubulin, Tuj1, to reveal axons) indicated that at E12.5 dorsal root axons were ensheathed exclusively by β-galactosidase-positive cells from their origin at the DREZ into the DRG (FIG. 3A-D). The cells extended long cytoplasmic processes (FIGS. 3A and 3B, arrows) between axons and surrounding perineurial cells (FIG. 3D, stars). β-galactosidase was never detected in the latter cells nor in cells within pial membranes (FIG. 3D and data not shown). The morphology of the β-galactosidase-positive cells and their tight association with axons identified them as Schwann cell precursors. Co-labelling for β-galactosidase and neuregulin receptor ErbB3, which is specifically expressed in PNS glial cells, confirmed their identity. Most, if not all, β-galactosidase positive cells positioned along dorsal roots were also ErbB3-positive (FIGS. 3E and 3F). Altogether, these observations suggest that DREZ BC cells give rise to the entire glial component of the dorsal roots.

BC Derivatives Contribute a Subset of Sensory Neurons and Satellite Cells to the DRG

In Krox20^{Cre/+},R26R embryos, at every axial level lacZ-expressing cells began to accumulate in the DRGs from E12 onwards (FIGS. 3 and 4A, and data not shown). These cells were mostly positioned on the medial side of the DRG, proximal to the spinal cord (FIG. 4A). Since BC-derived cells enter the DRG concomitant with the first appearance of postmitotic neurons, we investigated whether some BC cell derivatives adopt a neuronal fate. We found that many β-galactosidase-positive cells within the DRG also expressed the neuronal marker β-III-tubulin (FIGS. 4B and 4C, arrows). In addition, most of these cells expressed the NGF receptor TrkA (FIGS. 4D and 4E, arrows), present only in small-diameter neurons. Together these data show that some BC-derived cells invading the DRGs give rise to neurons. However, some of the β-galactosidase-positive cells in the DRG were β-III-tubulin-negative (FIGS. 4B and 4C, arrowheads), suggesting that part of the BC cell progeny in the embryonic DRGs might either remain uncommitted or adopt another fate, possibly glial (see below).

Since previous studies have shown that Krox20 is never expressed in DRG neurons or satellite cells^{6,13}, we were able to follow the fate of BC cell derivatives in adult DRGs. Lumbar (L3-L5) DRGs from Krox20^{lacZ/+} and Krox20^{Cre/+},R26R adult mice were dissected and stained with X-gal. Whilst in Krox20^{lacZ/+} animals no X-gal-positive cell body were found and labelling was restricted to Schwann cell processes extending into the DRG, in Krox20^{Cre/+},R26R DRGs a significant number of large cell soma was labelled (FIG. 5A). These were evenly distributed within the DRGs at different axial levels (data not shown).

To better characterise these cells, Krox20^{Cre/+},R26R ganglia were stained with Bluo-Gal, another substrate of β-galactosidase which generates an electron-dense blue precipitate. Examination of semi-thin sections revealed that the β-galactosidase-positive cells included cells with neuronal features, including relatively large cell bodies (mostly 15-20 μm in diameter, FIG. 5B). These cells represented approximately 5% of the total neurons in the DRG (80 positive cells were observed out of 1680 neurons in 4 DRGs at thoracic and lumbar levels). β-galactosidase-positive cells were then analysed on ultrathin sections by electron microscopy. We confirmed that most labelled cells displayed morphologies and sizes consistent with those of neuronal soma (FIG. 5C). In addition, we also identified small numbers of β-galactosidase-positive cells with much smaller nuclei and a spindle-shaped morphology that were closely associated with neuronal soma (FIG. 5D) and corresponded to satellite cells.

To confirm these findings using cell-type specific markers, we double-labelled sections of lumbar (L3-L5) DRG from adult Krox20^{Cre/+},R26R mice with antibodies directed against β-galactosidase and either NeuN, a neuron-specific marker or GFAP, a glial cell-specific marker. Consistent with our previous results, many lacZ-expressing cells were NeuN-positive (FIG. 5E). In addition, in accord with our electron microscopy analysis, small cells double positive for β-galactosidase and GFAP were found clustered in a ring-like arrangement around neurons (FIG. 5F).

Together, our data indicate that sub-populations of both satellite cells and neurons in the adult DRG are derived from Krox20-expressing BC cells. Although we cannot exclude the possibility that Schwann cells, that express Krox20 from E15.5 onwards, might contribute to these DRG populations, our analysis of embryonic DRGs strongly favours a BC-cell origin. Interestingly, β-galactosidase-positive satellite cells were often found in the vicinity of β-galactosidase-positive neurons (see for example FIG. 5F). These observations raise the possibility that these different cell types may have differentiated from a single pluripotent progenitor within the DRG.

Most BC-Derived Neurons in the DRG are Nociceptive Afferents

BC-derived cells first reach the DRGs during the period when nociceptive neurons are born (E10.5 to E13.5, ref.19). This raised the possibility that the BC-derived neurons in the DRGs might be predominantly nociceptive afferents. Consistent with this, we found that in E12.5 DRGs most β-galactosidase-positive neurons also expressed TrkA (FIGS. 4D and 4E), which is specific for nociceptive afferents 20. Also, the size range of BC-derived neurons in adult DRGs measured in semi-thin sections fell within the small-to-medium diameter range (rarely exceeding 30 μm; FIG. 5B). To confirm their nociceptive phenotype, we performed co-labelling in adult L3-L5 DRGs for β-galactosidase and markers of the two major types of nociceptive afferents, calcitonin-gene related peptide (CGRP) and isolectin B4 (IB4). These markers are restricted to non-overlapping populations of adult DRG neurons, defining peptidergic and non-peptidergic nociceptors, respectively^{21,22}. 40% of the β-galactosidase-positive cells with neuronal morphology were co-labelled with IB4 (n=140; FIGS. 6A and 6B) and 47% co-expressed CGRP (n=105; FIGS. 6C and 6D). Therefore, we estimate that at least 87% of the β-galactosidase-positive neurons are nociceptive afferents. We then tried to characterise the remaining β-galactosidase-positive neurons by double labelling with parvalbumin, a marker of proprioceptive DRG neurons^{23,24}. Only about 1% of the β-galactosidase-positive neurons expressed parvalbumin (n=382; FIGS. 6E and 6F), whereas the frequency of parvalbumin-positive cells is 10 to 15% in the DRG. (data not shown and ref. 25). Altogether these data indicate that the vast majority of BC derivatives that adopt a neuronal fate in the DRG become nociceptive neurons.

Interestingly, we also found that β-galactosidase-positive satellite cells were preferentially associated with large diameter proprioceptive neurons. 45% of the neurons in contact with β-galactosidase-positive satellite cell(s) (n=44) were parvalbumin-positive, and 89% were large diameter neurons (see FIGS. 5F and 6F as examples).

Purification of BC Cells

Mammalian BC cells were isolated isolated from Krox20 knock-in mouse embryos, i.e. Krox₂₀^{lacZ/+(11)} or Krox₂₀^{GFP(DT)/+(14)} embryos, using a Fluorescence Activated Cell Sorter (FACS-Gal⁽⁴¹⁾ or FACS procedure, respectively).

Krox20^{lacZ/+} E12.5 embryos are genotyped based on β-glactosidase activity in cranial BC cells, as revealed with a fluoregenic substrate (FDG, Sigma F-2756), using a fluorescence microscope. Krox20^{GFP/+} E12.5 embryos are genotyped based on endogeneous fluorescence in cranial BC cells, using a fluorescence microscope.

Embryos are stored and dissected in L15 medium (Gibco) at 4° C. Heads are cut and discarded. The neural tube is opened dorsally using microscissors, and the skin in separated from the neural tube. The neural tube, with dorsal roots, ventral roots and DRG still attached to it, is separated from the rest of the embryo using sharp forceps. Pial membranes with attached roots and DRGs are finally separated from the neural tube with sharp forceps. Because BC cells remain associated with the pial membranes, these dissection steps allow us to enrich BC cells in the dissected tissue. Pial membranes with attached roots and DRGs are dissociated in 100 μg/ml hyaluronidase (Sigma)/200 μg/ml collagenase (Sigma)/Hank's buffer (Gibco) for 10 minutes at 37° C. Dissociation is stopped by repeated dilution in DMEM (Gibco). Cells are then dissociated mechanically by pipeting, centrifugated (1800 rpm, 2 min), rinsed twice in Hank's buffer and sorted on a Facsort (Beckton-Dickinson) immunocytometry system. 2.10⁴ purified BC cells are typically obtained from 1 embryo.

Identification of BC Cells in the Human Foetus

Human fetuses (between 7 and 8 weeks of pregnancy) were analyzed. The fixed tissues were obtained from a specialized medical center, and were abortion materials. Spinal cords with attached spinal roots and DRGs were dehydrated and embedded in paraffin. 10 μm sections were cut and mounted on Superfrost glass/plus slides (Menzel Glaser). Sections were dewaxed, rehydrated, incubated with a blocking solution (3% BSA/0.1% Triton/PBS) for 1 hr at RT, and finally incubated with an anti-Krox20 polyclonal antibody (BabCO, 1/500) diluted in 1% BSA/0.1% Triton/PBS. The primary antibody was revealed using an alkaline phosphatase (AP)-conjugated anti-rabbit secondary antibody. Sections were finally counterstained, dehydrated and mounted with Eukit.

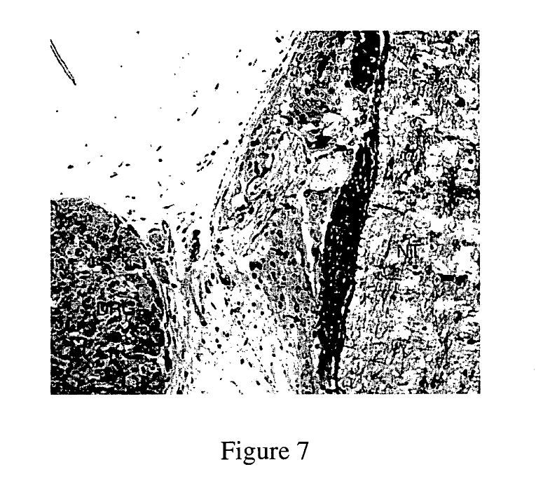

Densely packed clusters of cells were present at nerve entry and exit points, adjacent to the spinal cord. The morphology and position of these cells were consistent with a BC cell identity. Moreover most of these cells were labeled with an antibody directed against Krox20, a known BC-marker in the mouse (see FIG. 7). This examples thus clearly reveals, for the first time, the existence of a population of cells in the human with cellular and molecular features consistent with a BC identity.

### REFERENCES

1. Le Douarin, N. M. (1982). The neural crest (Cambridge University Press, Cambridge, UK.).

2. Farinas, I., Cano-Jaimez, M., Bellmunt, E. & Soriano, M. Regulation of neurogenesis by neurotrophins in developing spinal sensory ganglia. Brain. Res. Bull. 57, 809-816 (2002).

3. Snider, W. D. & Wright, D. E. Neurotrophins cause a new sensation. Neuron 16, 229-232 (1996).

4. Dupin, E., Baroffio, A., Dulac, C., Cameron-Curry, P. & Le Douarin, N. M. Schwann-cell differentiation in clonal cultures of the neural crest, as evidenced by the anti-Schwann cell myelin protein monoclonal antibody. Proc. Natl. Acad. Sci. USA 87, 1119-1123 (1990).

5. Hagedorn, L. et al The Ets domain transcription factor Erm distinguishes rat satellite glia from Schwann cells and is regulated in satellite cells by neuregulin signaling. Dev. Biol. 219, 44-58 (2000).

6. Murphy, P. et al. The regulation of Krox-20 expression reveals important steps in the control of peripheral glial cell development. Development 122, 2847-2857 (1996).

7. Niederlander, C. & Lumsden, A. Late emigrating neural crest cells migrate specifically to the exit points of cranial branchiomotor nerves. Development 122, 2367-2374 (1996).

8. Altman, J. & Bayer, S. A. Development of the cranial nerve ganglia and related nuclei in the rat. Adv. Anat. Embryol. Cell. Biol. 74, 1-90 (1982).

9. Altman, J. & Bayer, S. A. The development of the rat spinal cord. Adv. Anat. Embryol. Cell. Biol. 85, 1-164 (1984).

10. Golding, J. P. & Cohen, J. Border controls at the mammalian spinal cord: late-surviving neural crest boundary cap cells at dorsal root entry sites may regulate sensory afferent ingrowth and entry zone morphogenesis. Mol. Cell. Neurosci. 5, 381-396 (1997).

11. Schneider-Maunoury, S. et al. Disruption of Krox-20 results in alteration of rhombomeres 3 and 5 in the developing hindbrain. Cell 75, 1199-1214 (1993).

12. Wilkinson, D. G., Bhatt, S., Chavrier, P., Bravo, R. & Charnay, P. Segment-specific expression of a zinc-finger gene in the developing nervous system of the mouse. Nature 337, 461-464 (1989).

13. Topilko, P. et al. Krox-20 controls myelination in the peripheral nervous system. Nature 371, 796-799 (1994). 4-8 Sep. 2001: Poster, MRC Centre for Developmental Neurobiology, inaugural conference, KCL, London, UK; 26 Feb. 2003: Oral communication+ Poster, Glial cell club, London, UK; 14-17 Jun. 2003: Oral communication, Boundaries in development: 30 years of progress, EMBL, Heidelberg, Germany: “Boundary caps: a gating function at the CNS/PNS interface and a source of pluripotent neural cells in the PNS”; 14-18 Jun. 2003: Oral communication, Conference Jacques Monod, La Londe-les maures, France: “Boundary cap cells play unexpected roles during PNS development”; 22-27 Jun. 2003: Poster, Gordon Conference, Developmental Biology, Andover, N.H., USA: “Boundary cap cells give rise to sensory neurons and glia in the developing peripheral nervous system”; 1-3 Oct. 2003: Poster, Ascona, Switzerland: “Boundary cap cells give rise to sensory neurons and glia in the developing peripheral nervous system”.

14. Vermeren, M. et al. Integrity of developing spinal motor columns is regulated by neural crest derivatives at motor exit points. Neuron 37, 403-415 (2003).

15. Voiculescu, O., Charnay, P. & Schneider-Maunoury, S. Expression pattern of a Krox-20/Cre knock-in allele in the developing hindbrain, bones, and peripheral nervous system. Genesis 26, 123-126 (2000).

16. Voiculescu, O. et al. Hindbrain patterning: Krox2O couples segmentation and specification of regional identity. Development 128, 4967-4978 (2001).

17. Soriano, P. Generalized lacZ expression with the ROSA26 Cre reporter strain. Nat. Genet. 21, 70-71 (1999).

18. Rosario, C. M. et al. Differentiation of engrafted multipotent neural progenitors towards replacement of missing granule neurons in meander tail cerebellum may help determine the locus of mutant gene action. Development 124, 4213-4224 (1997).

19. Lawson, S. N. & Biscoe, T. J. Development of mouse dorsal root ganglia: an autoradiographic and quantitative study. J. Neurocytol. 8, 265-274 (1979).

20. Mu, X., Silos-Santiago, I., Carroll, S. L. & Snider, W. D. Neurotrophin receptor genes are expressed in distinct patterns in developing dorsal root ganglia. J Neurosci. 13, 4029-4041 (1993).

21. Snider, W. D. & McMahon, S. B. Tackling pain at the source: new ideas about nociceptors. Neuron 20, 629-632 (1998).

22. Molliver, D. C. et al. IB4-binding DRG neurons switch from NGF to GDNF dependence in early postnatal life. Neuron 19, 849-861 (1997).

23. Arber, S., Ladle, D. R., Lin, J. H., Frank, E. & Jessell, T. M. ETS gene Er81 controls the formation of functional connections between group Ia sensory afferents and motor neurons. Cell 101, 485-498 (2000).

24. Honda, C. N. Differential distribution of calbindin-D28k and parvalbumin in somatic and visceral sensory neurons. Neuroscience 68, 883-892 (1995).

25. Ernfors, P., Lee, K. F., Kucera, J. & Jaenisch, R. Lack of neurotrophin-3 leads to deficiencies in the peripheral nervous system and loss of limb proprioceptive afferents. Cell 77, 503-512 (1994).

26. Sharma, K., Korade, Z., and Frank, E. Late-migrating neuroepithelial cells from the spinal cord differentiate into sensory ganglion cells and melanocytes. Neuron 14, 143-152 (1995).

27. Nakagawa, S. & Takeichi, M. Neural crest emigration from the neural tube depends on regulated cadherin expression. Development 125, 2963-2971 (1998).

28. Patel, T. D. et al. Peripheral NT3 signaling is required for ETS protein expression and central patterning of proprioceptive sensory afferents. Neuron 38, 403-416 (2003).

29. Dong, X., Han, S., Zylka, M. J., Simon, M. I. & Anderson, D. J. A diverse family of GPCRs expressed in specific subsets of nociceptive sensory neurons. Cell 106, 619-632 (2001).

30. Anderson, D. J. Lineages and transcription factors in the specification of vertebrate primary sensory neurons. Curr. Opin. Neurobiol. 9, 517-524 (1999).

31. Ma, Q., Fode, C., Guillemot, F. & Anderson, D. J. Neurogeninl and neurogenin2 control two distinct waves of neurogenesis in developing dorsal root ganglia. Genes Dev. 13, 1717-1728 (1999).

32. Morrison, S. J. Neuronal potential and lineage determination by neural stem cells. Curr Opin Cell Biol 13, 666-672 (2001).

33. Morrison, S. J. et al Transient Notch activation initiates an irreversible switch from neurogenesis to gliogenesis by neural crest stem cells. Cell 101, 499-510 (2000).

34. Wakamatsu, Y., Maynard, T. M. & Weston, J. A. Fate determination of neural crest cells by NOTCH-mediated lateral inhibition and asymmetrical cell division during gangliogenesis. Development 127, 2811-2821 (2000).

35. Morrison, S. J., White, P. M., Zock, C. & Anderson, D. J. Prospective identification, isolation by flow cytometry, and in vivo self-renewal of multipotent mammalian neural crest stem cells. Cell 96, 737-749 (1999).

36. Hagedorn, L., Suter, U. & Sommer, L. P0 and PMP22 mark a multipotent neural crest-derived cell type that displays community effects in response to TGF-beta family factors. Development 126, 3781-3794 (1999).

37. Itasaki, N., Bel-Vialar, S. & Krumlauf, R. ‘Shocking’ developments in chick embryology: electroporation and in ovo gene expression. Nat. Cell Biol. 1, E203-207 (1999).

38. Wilkinson, D. G. Whole-mount in situ hybridisation of vertebrate embryos. In In Situ Hybridisation: A practical Approach (ed. D. G. Wilkinson), pp 75-83., Oxford: IRL Press, 1992).

39. Weis, J., Fine, S. M., David, C., Savarirayan, S. & Sanes, J. R. Integration site-dependent expression of a transgene reveals specialized features of cells associated with neuromuscular junctions. J. Cell Biol. 113, 1385-1397 (1991).

40. Vitalis, T., Alvarez, C., Chen, K., Shih, J. C., Gaspar, P. & Cases, O. Developmental expression of monoamine oxidases in sensory organs and neural crest derivatives. J. Comp. Neurol. 464, 392-403 (2003).

41. Fiering, S. N., Roederer, M., Nolan, G. P., Micklem, D. R., Parks, D. R. & Herzenberg, L. A. Improved FACS-Gal: flow cytometric analysis and sorting of viable eukaryotic cells expressing reporter gene constructs. Cytometry 12(4), 291-301 (1991).

1. A method of producing neural pluripotent cells, the method comprising the ex vivo or in vitro culture, under suitable conditions, of mammalian Boundary Cap cells.

2. A method of producing differentiated cells, the method comprising the ex vivo or in vitro culture of mammalian Boundary Cap cells or their progeny, under conditions suitable for differentiation of said cells.

3. The method of claim 2, wherein the BC cells are cultured in the presence of differentiation factors selected from trophic factors, stem cell factors, colony-stimulating factors and lymphokines.

4. The method of claim 2, wherein differentiation into distinct cell types is detected using cell-specific makers.

5. A pharmaceutical composition comprising BC cells, their progeny or derivatives thereof, and a suitable vehicle, excipient or carrier.

6. The pharmaceutical composition of claim 5, wherein said composition comprises from 10² to 10⁶ cells or more.

7. A method of tissue re-engineering, comprising administering to a subject in need thereof a suitable amount of mammalian BC cells or their progeny, under conditions allowing differentiation or migration of said cells in said subject, thereby allowing tissue re-engineering.

8. The method of claim 7, wherein said conditions include the administration in, at or near a site of neurological disorder.

9. The method of claim 8, wherein said subject suffers from peripheral nerve demyelination, injury or degeneration and wherein said cells are administered to the patient at a site of demyelination, injury or degeneration.

10. A method of claim 7 for reconstituting neural tissue in a subject, comprising administering to a subject in need thereof a suitable amount of mammalian BC cells or their progeny, under conditions suitable for differentiation of said cells into differentiated neural cells in said subject, thereby allowing tissue reconstitution.

11. A method of treating, reducing or alleviating pain in a subject, the method comprising administering to the subject an amount of mammalian BC cells or their progeny, under conditions allowing said cells to differentiate into nociceptive neurons in said subject.

12. A method of treating, reducing or alleviating pain in a subject, the method comprising culturing mammalian BC cells or their progeny in vitro or ex vivo under conditions allowing said cells to differentiate into nociceptive neurons, and administering to the subject said nociceptive neurons.

13. The method of claim 7 or 11 wherein the mammalian BC cells are autologous with respect to the subject.

14. A method of screening compounds that modulate neuronal cell migration and/or differentiation, the method comprising contacting a candidate compound with a BC cell or a progeny thereof, and determining whether said compound modulates migration and/or differentiation of said cells.

15. The method of claim 14, wherein said contacting is performed in vitro or ex vivo.

16. The method of claim 14, wherein said contacting is performed in vivo in a non-human animal.

17. The method of claim 14, for screening compounds that modulate neuron cell differentiation, the method comprising contacting a candidate compound with a BC cell or a progeny thereof, and determining whether said compound modulates differentiation of said cells into cells expressing a neuronal marker selected from the group of β-III tubulin, NeuN, NGF receptor TrkA and parvalbumin.

18. The method of claim 14, for screening compounds that modulate glial cell differentiation, the method comprising contacting a candidate compound with a BC cell or a progeny thereof, and determining whether said compound modulates differentiation of said cells into cells expressing a glial cell marker selected from the group of GFAP and ErbB3.

19. The method of claim 14, for screening compounds that modulate nociceptive cell differentiation, the method comprising contacting a candidate compound with a BC cell or a progeny thereof, and determining whether said compound modulates differentiation of said cells into cells expressing a nociceptive cell marker selected from the group of NGF receptor TrkA, Calcitonin-Gene Related Peptide (CGRP) and Isolectin B4 (IB4).

20. The method of claim 14, for screening compounds that modulate proprioceptive cell differentiation, the method comprising contacting a candidate compound with a BC cell or a progeny thereof, and determining whether said compound modulates differentiation of said cells into cells expressing a proprioceptive cell marker selected from parvalbumin.

21. The method of claim 14, for screening compounds that modulate satellite cell differentiation, the method comprising contacting a candidate compound with a BC cell or a progeny thereof, and determining whether said compound modulates differentiation of said cells into cells expressing a satellite cell marker, selected from GFAP.
